(19) United States
(12) Patent Application Publication
(10) Pub. No.: US 2013/0137988 A1
(43) Pub. Date: May 30, 2013

(54) Method and Apparatus for the Augmentation of Physical Examination over Medical Imaging Data

(71) Applicants: Orna Bregman-Amitai, Tel-Aviv (IL); Eyal Toledano, Kiryat Ata (IL); Eduard Oks, Bat-Yam (IL); Ronnie Tepper, Herzelia (IL)

(72) Inventors: Orna Bregman-Amitai, Tel-Aviv (IL); Eyal Toledano, Kiryat Ata (IL); Eduard Oks, Bat-Yam (IL); Ronnie Tepper, Herzelia (IL)

(73) Assignee: SAMSUNG ELECTRONICS CO., LTD., Gyeonggi-do (KR)

(21) Appl. No.: 13/304,717

(22) Filed: Nov. 28, 2011

(51) Int. Cl. A61B 8/00

(52) U.S. Cl. 600/459

(57) ABSTRACT

An augmented ultrasound examination system, comprises: a) an ultrasound system suitable to generate images of a body portion; b) a first position sensor coupled to the ultrasound transducer of said ultrasound system; c) a second position sensor suitable to be coupled to a finger; and d) data processing apparatus suitable to receive position information from said first and from said that second position sensors and to generate therefrom information correlating on a screen the position of said second position sensor with the image generated by said ultrasound system.

### FIELD OF THE INVENTION

The present invention relates to ultrasound medical devices. More particularly, the invention relates to the correlation of ultrasound images with physical hand positioning.

### BACKGROUND OF THE INVENTION

Ultrasound is frequently used during various diagnostic procedures. In some countries the utrasound process in done in two stages: in the first stage the technician is responsible for acquisition of the images, and in the second stage the physician is responsible for the offline diagnosis. Ultrasound is often followed by a physical examination in which the physician touches and senses the relevant body part.

Currently, there is no existing tool that enables the physician to accurately know, by viewing in real time, the exact point he is touching in a body cavity, relative to the ultrasound view. Thus, for example, in gynecological physical examination, the physician relies on his sense of touch only, and cannot know where his hand is in real time, relative to the inner body organs that he may have seen before in the ultrasound. While sensing the kidney or liver, the physician can only assume that he remembers the ultrasound image he saw before and the previous location of the transducer.

In spite of the broad use of ultrasound in the art, there is no known solution to the specific problem described above. Various solutions have been suggested instead for the registration of imaging data in known medical procedures. For example: Mediguide developed sensors for intrabody positioning system for medical devices, designed for guide-wires, coronary catheterization devices, and the like (http://medgadget.com/search/mediguide). U.S. Patent Application No. 2011/0040175 deals with the real-time positioning (location and orientation) of surgical instruments during operation, with reference to ultrasound transducers. U.S. Pat. No. 7,824,328 teaches the positioning of endoscopes during operation. U.S. Pat. No. 7,662,113 teaches the tracking of fingers during physical examination for training purposes, without registration with imaging data and with no integration of the information with the ultrasound information.

Therefore a need exists for systems that will allow displaying the location of a physician's finger over imaging data during physical examination. It is therefore an object of the present invention to provide a method and system that will allow the physician to perceive in real time how the image he views on a screen correlates with the position of his hand or finger and the tactile information he derives from them.

### SUMMARY OF THE INVENTION

The invention relates to an augmented ultrasound examination system, comprising:
a) an ultrasound system suitable to generate images of a body portion;
b) a first position sensor coupled to the ultrasound transducer of said ultrasound system;
c) a second position sensor suitable to be coupled to a finger; and
d) data processing apparatus suitable to receive position information from said first and from said that second position sensors and to generate therefrom information correlating on a screen the position of said second position sensor with the image generated by said ultrasound system.

In one embodiment of the invention said first position sensor comprises a plurality of sensors. In another embodiment of the invention said second position sensor comprises a plurality of sensors.

The augmented system of the invention may further comprise registration markers, either external or such that are suitable to be applied to a patient's body.

In another aspect the invention is directed to a method for generating augmented ultrasound examination system, comprising:
a) providing an ultrasound system suitable to generate images of a body portion;
b) providing a first position sensor coupled to the ultrasound transducer of said ultrasound system;
c) providing a second position sensor suitable to be coupled to a finger;
d) providing data processing apparatus suitable to receive position information from said first and from said that second position sensors; and
e) correlating on a screen the position of said second position sensor with the image generated by said ultrasound system, using the data received in said data processing apparatus.

### BRIEF DESCRIPTION OF THE DRAWINGS

In the drawings:

FIG. 1 illustrates how during vaginal physical examination a physician can see his finger's location (red dot) in real time over the ultrasound data acquired earlier, according to one embodiment of the invention;

FIG. 2 is a schematic description of a system according to one embodiment of the invention;

FIG. 3 is a schematic description of a system according to an embodiment of the invention, with sensors adjusted to the finger of the physician, which allows him to wear gloves while enabling full sensing capabilities;

FIG. 4 illustrates the use of an optional display during a physical examination, according to another embodiment of the invention; and

FIG. 5 is a schematic description of an alternative positioning arrangement of a single sensor (FIG. 5A) and of multiple sensors (FIG. 5B) on a physician's hand.

### DETAILED DESCRIPTION OF THE INVENTION

Physicians are used to look at special displays (used in ultrasound apparatus) while carrying out examinations. A display that provides the physician with information about the relative position of their fingers to the object under examination is needed to improve the diagnostic accuracy.

The need for an augmented display is even higher when:
a. The ultrasound was acquired by a different person than the one who does the physical examination. In this case, the probability of incorrectly identifying the touched location relative to the image is high, thus resulting in an inaccurate diagnosis.
b. Performing a gynecological physical examination where the physician cannot see where the ultrasound transducer was and where are their fingers. In this case the touch sense is not related to the ultrasound information.

The invention provides a system that enables a physician to see the position of his fingers over an ultrasound display during physical examinations or other procedures. Positioning sensors are used, in addition to the ultrasound transducer and to the fingers of the physician during the physical examination, in order to enable the integrated display.

According to the invention the following is provided:
1. Markers placed on the patient's body (or on the examination environment if the patient does not move), which define the coordinate system;
2. Positioning sensors added to the transducer, which permit the registration of the ultrasound information in the said coordinate system;
3. One or more sensors provided on the physician's fingers, which permits to register the fingers' movement to the same coordinate system;
4. A common real-time display of the acquired ultrasound and the real-time finger(s) position(s).

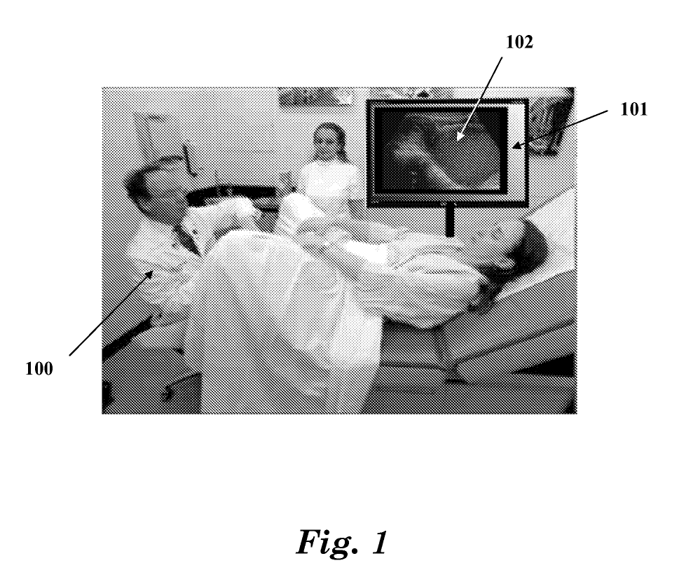

Referring now to FIG. 1, the situation resulting from the operation of an embodiment of the invention is illustrated. In this scenario the physician 100 can see on the screen 101 his finger's location (not shown) superimposed over the ultrasound information 102 in real time, during the physical examination.

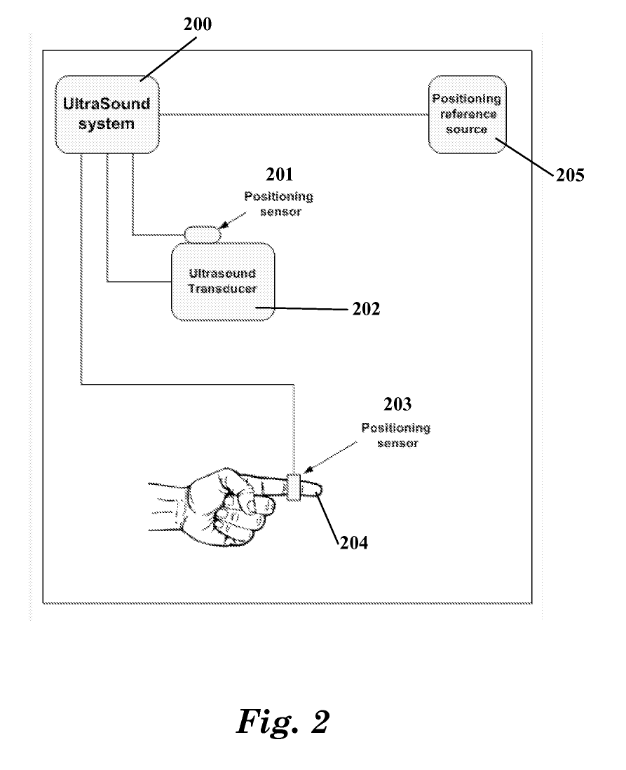

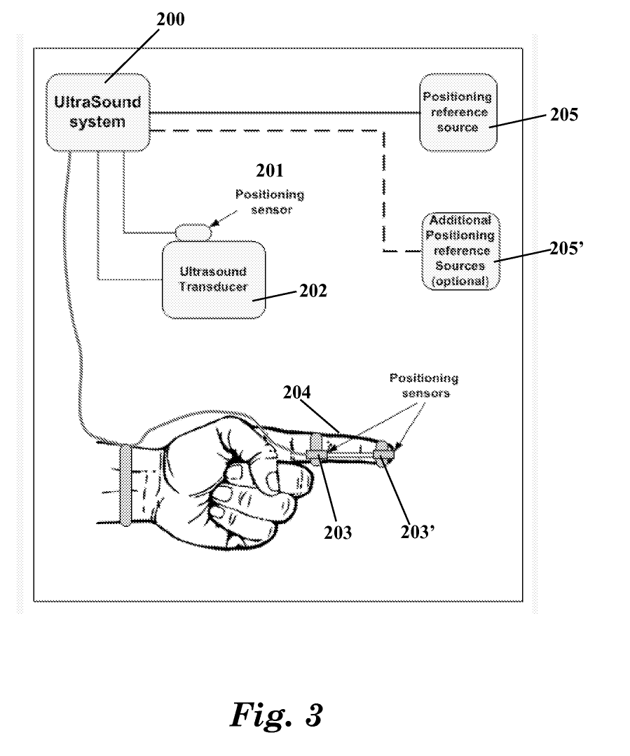

Looking now at FIG. 2, a schematic description of a basic system according to one embodiment of the invention is provided, which is composed of an ultrasound positioning unit comprising a positioning sensor 201 coupled to the ultrasound transducer 202 of ultrasound system 200, and another sensor 203 coupled to the physician's finger 204. A positioning reference source 205 establishes the relative position of the positioning sensors relative to an arbitrary coordinate system. As will be apparent to the skilled person, although the figure shows only one sensor coupled to the transducer and one coupled to the physician's hand, more than one transducer can be coupled to either or both locations, to increase accuracy or to acquire additional information. The figure illustrates the most basic setup, for the purpose of clarity, but of course the invention is not limited to such basic setup. For instance, as shown in FIG. 3 (which uses the same reference numbers as FIG. 2, for clarity), which is an alternative schematic description of a system according to another embodiment of the invention with sensors attached to the finger 204 of the physician, two sensors 203 and 203′ are placed on the finger of the examining physician and, still alternatively, one sensor can be turned to the other side of the finger to improve accuracy. This setup can be used where the physician wears glove. It enables the system to bring finger tip accuracy to the interaction. Additional synchronized reference sources, such as source 205′, can be added in order to extend the range or the accuracy of the system. Using a two sensors constellation allows six degrees of freedom positioning capabilities. In any case, as will be apparent to the skilled person the added sensors should not interfere with the physician's work while conducting the examination and their position on the fingers should be optimized to enable sensing while touching. An alternative implementation for this system is by using two needle-thick sensors (or a single sensor) that is attached to the physician's hand. The exact location of the positioning sensors can be altered according to the physician's actual needs and the type of physical examination.

Although in the examples provided herein reference is made to magnetic positioning sensors, the specific type of sensors is only mentioned for the purpose of illustration and is not intended to limit the invention in any way. As will be easily appreciated by persons skilled in the art, any other suitable positioning sensor, such as, e.g., Wi-Fi or radio based positioning sensors, can be used, without exceeding the scope of the invention.

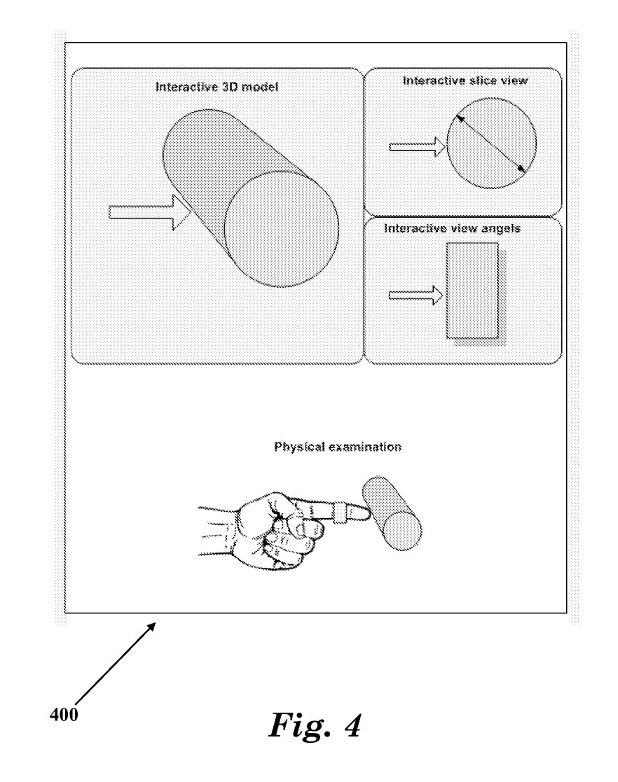

The invention permits to provide enhanced displaying capabilities, because of the augmented data made available to it. For the purpose of illustration FIG. 4 shows an optional display that can be made available to the physician during the physical examination, which is self-explanatory.

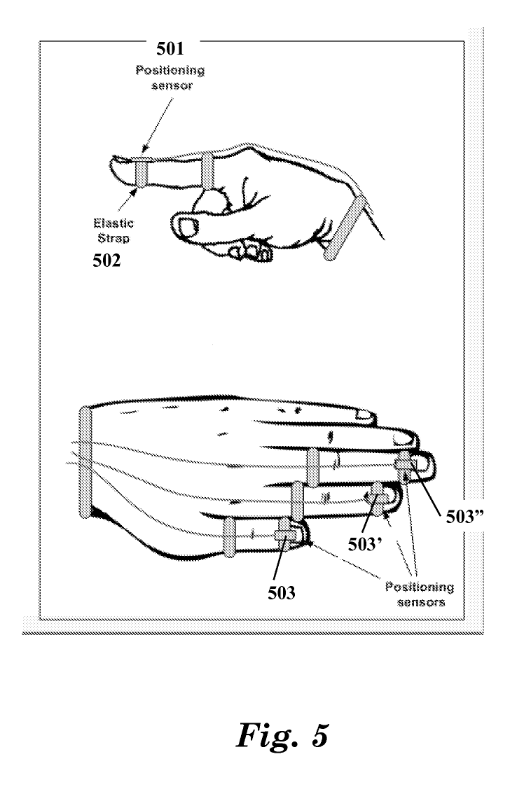

FIG. 5 schematically illustrates a possible positioning of a single sensor 501, using, e.g., an elastic strap 502 (FIG. 5A) and of multiple sensors 503, 503′ and 503″ (FIG. 5B) on a physician's hand.

The positioning sensor must be coupled to the transducer in a way that the coupling is fixed so that the sensors measurement is not effected by the transducer's activity. For example, if sensors are employed, which use magnetic fields for calculations, with a transducer that uses a mechanical engine for 3D/4D acquisition, then the sensor must be located far from the engine so that the magnetic noise created by the engine will not affect the measurements. The system should be designed in such a way that will enable the location of the volume in the sensors coordinate system to be measured.

Positioning markers adjusted to patient are needed when there is a chance that the patient will move between the ultrasound and the physical examination. If the patient will not move (i.e., will not perform a movement that may affect the required accuracy of the physical examination), the positioning markers will not add information and therefore the operation can be performed without them. Yet, it is often useful to employ markers to verify that no movement is taking place.

Positioning markers should be applied to the patient in a way that repeating the registration process gives same results. There are mainly two types of markers:
1. Sensors that are adjusted to the patient's body—in this case the registration process is automatic and there is no need for a registration procedure. The number of required markers depends on the sensors—accuracy, number of degree of freedom etc'. For example—one 6DOF (i.e., 6 Degrees of Freedom) such as the Patriot™ manufactured by Polehemus (http://polhemus.com/?page=Motion_PATRIOT) marker can give full registration information; using 5DOF requires at least 2 sensors with no common rotation axis. The sensors adjustment should be to body parts that remain rigid in reference to the organs under examination. For example, it is useful to attach the reference positioning sensor to the low back in case of vaginal examination, but that location is not relevant for examination of the breast.
2. Non-sensors markers on patient's body—in this case there is a need to apply a registration procedure where another sensor is used to enter the position of the marker. Such a procedure can involve touching the markers with the transducer or finger with sensor. The markers are 3DOF markers and therefore at least 3 markers are needed for registration. The registration procedure must be performed before the ultrasound images are acquired, and again before the physical examination or any other procedure is carried out.

It is usually desirable to use more markers than the minimum for verification of the registration. If the registration failed at any stage (manual or automatic), the physician must be notified. While using sensors with lower accuracy than required, the accuracy can be improved when using multiple sensors.

There is no change in the acquisition procedure of US/Doppler 2D/3D/4D data and the procedure can be performed with any US (ultrasound) device and transducers. The process may include acquisition of multiple 2D/3D/4D gray/Doppler data. The system will pose all data in the same coordinate system, thus enabling a location-based search of the data.

### Pre-physical Examination

The physical examination can be applied in a place where the ultrasound was acquired or elsewhere. In both cases, the patient's position should not be changed in a way that may cause deformation of the relevant body part.

### Adjusting Sensors to the Physician Fingers

As explained above, the physician can use a one-finger sensor or a plurality of sensors. He may also have sensors on both hands. The sensors and/or the glove must not interfere with the physical examination or affect the physician's touching sense after short adaption period. The sensors can be wired together, or separately, to a central source or can use wireless communication to publish their positions.

### Physician's Finger Calibration

The main use of a finger sensor is to display the position over the ultrasound information. The system can work well while showing the location of the sensors (rather than the finger tip) and leaving the physician to estimate the position correction in his head. However, using a simple calibration process will enable the system to display the exact position of the finger tips or the finger volume on screen.

There are many valid methods to apply such calibration. For example, touching a dedicated sensor or touching a marker with known position. Another simple calibration method (suitable only when both fingers have sensors) is meeting the thumb and the index finger, and dividing the distance by 2. The accuracy of this simple method is limited but may be good enough for many purposes.

It is recommended to repeat the calibration every time the physician wears the sensors, but when the required accuracy is limited, the calibration can be performed only on the first use by the physician.

### Selecting the Displayed Ultrasound Intersection

The physician should select the intersection and/or display mode of the ultrasound in order for the process to be efficient, and in order for him to control it during the examination. The ultrasound display option can be the same as the existing display options of the ultrasound. In case of invasive physical examination (gynecology or other), the display may be according the finger location, enabling the physician to see the environment of the finger. In the case of multiple data of the same location, the user may select which data to display or multiple display option.

As will be apparent from the above description, the invention provides a tool and method, which enable the physician to “see” where he is sensing during the physical examination, relative to the ultrasound information. It enhances the physical examination with visual information from the ultrasound. The interactive ultrasound information can be stored along with the positioning information so that the physical examination can be repeated using the already stored ultrasound data. This permits to enhance the ultrasound capabilities in cases of a second opinion, and expert examination, follow-up examinations, and more.

All the above description and examples of use have been provided for the purpose of illustration and are not intended to limit the invention in any way. Many different systems and setups can be devised, using many different sensors and elements, all without exceeding the scope of the invention.

1. An augmented ultrasound based invasive examination system, comprising:
a) an ultrasound system for generating images of an intrabody portion;
b) two or more registration markers, appliable to a patient's body, which together provide registration data for defining a coordinate system along six degrees of freedom;
c) a first position sensor coupled to an ultrasound transducer of said ultrasound system, for registering said generated images in said coordinate system;
d) one or more second position sensors coupleable to a finger, for registering movements of said finger to said coordinate system;
e) a corresponding element for coupling each of said one or more second position sensors to said finger, wherein the overall thickness of said finger, said one or more second position sensors, and said coupling element is less than an outer dimension of a body lumen into which said finger is insertable during performance of an invasive physical examination of said intrabody portion, permitting to receive tactile information from said intrabody portion by said finger while viewing said generated ultrasound images; and
f) data processing apparatus operable to receive position information from said first and one or more second position sensors and to generate therefrom information correlating on a screen the position of said one or more second position sensors with the image generated by said ultrasound system.

2. The augmented system according to claim 1, wherein said first position sensor is embodied by a plurality of sensors.

3-5. (canceled)

6. A method for performing an augmented ultrasound based invasive examination, comprising:
a) providing an ultrasound system comprising an ultrasound transducer, for generating images of an intrabody portion;
b) providing a first position sensor coupled to the ultrasound transducer of said ultrasound system;
c) providing one or more second position sensors coupled to a finger;
d) providing data processing apparatus for receiving position information from said first and one or more second position sensors;
e) performing an invasive physical examination by touching said intrabody portion with the finger to which is coupled said one or more second position sensors; and
f) correlating on a screen the position of said one or more second position sensors with the image generated by said ultrasound system, using the data received in said data processing apparatus.

7. The method according to claim 6, wherein tactile information is received from said intrabody portion by said finger while viewing said generated ultrasound images.

8. The method according to claim 7, wherein the tactile information is received from said intrabody portion by said finger while viewing said generated ultrasound images in real time.

9. The method according to claim 7, further comprising the steps of storing the generated ultrasound images and the position information from the first position sensor in memory, receiving tactile information from the intrabody portion by the finger to which is coupled the one or more second position sensors, and viewing a common display of the stored ultrasound images and of real time position information of the one or more second position sensors.

10. The method according to claim 9, wherein the ultrasound images are generated by a first medical practitioner and the invasive physical examination is performed by a second medical practitioner.

11. The method according to claim 6, further comprising the step of calibrating a spacing between one of the second position sensors and the finger tip, whereby to correlate on the screen the position of the finger tip with the image generated by said ultrasound system.

12. The augmented system according to claim 1, wherein the two or more registration markers are sensors.

13. The augmented system according to claim 1, wherein the coupling element is a strap.
